(19) United States
(12) Patent Application Publication
(10) Pub. No.: US 2003/0059378 A1
(43) Pub. Date: Mar. 27, 2003

(54) Method of preventing mucositis

(76) Inventor: Barry M. Libin, Great Neck, NY (US)

Correspondence Address: James V. Costigan, Esq., HEADMAN & COSTIGAN, P.C., Suite 2003, 11185 Avenue of the Americas, New York, NY, 10036-2646, US

(21) Appl. No.: 09/948,416

(22) Filed: Sep. 6, 2001

(51) Int. Cl. A61K 7/16; A61K 31/075; A61K 31/44

(52) U.S. Cl. 424/49; 514/358; 514/721

(57) ABSTRACT

A method of preventing mucositis of mucosal tissues in patients is disclosed which is based on contacting the affected area with an amount of composition which comprises triclosan in amounts which are effective to prevent the symptoms of mucositis.

### BACKGROUND OF THE INVENTION

As disclosed in U.S. Pat. No. 5,945,089, immunodeficient patients frequently exhibit a condition of the mucosal tissues which is clinically described as mucositis. This condition has no known microbial or viral vector that has been implicated as the causative agent. The immunodeficiency that preceded the appearance of mucositis may arise spontaneously from genetic factors, may be caused by infections, e.g., the HIV virus or mucositis be induced as a result of chemotherapy or radiation therapy for neoplastic diseases. This condition has been difficult to treat and has not satisfactorily responded to treatment with antimicrobial or any other agents.

The applicant has discovered that mucositis may be prevented by contacting mucosal tissues with either triclosan alone or a combination of triclosan and a cationic agent. The present inventor holds U.S. Pat. No. 5,236,699, which is incorporated by reference. That patent describes the use of a mouth rinse which contains triclosan and a cationic antibacterial agent for use inter alia the treatment of plaque and gum diseases.

### SUMMARY OF THE INVENTION

The present invention comprises a method of preventing mucositis which comprises applying to the mucosal tissues an effective amount of a composition which comprises triclosan or a combination of triclosan and a cationic agent.

It is a primary object of the invention to provide a method for the prevention of mucositis using either triclosan or a combination of triclosan and a cationic agent in the oral, pharyngeal, esophageal, gastrointestinal and other mucosal tissues.

It is also a primary object of the invention to provide a method for the prevention of mucositis in the oral, pharyngeal, esophageal, gastrointestinal and other mucosal tissues in immunocompromised patients.

These and other objects of the invention will become apparent from a review of the appended specification.

### DETAILED DESCRIPTION OF THE PRESENT INVENTION

Mucositis is prevented in accordance with the present invention by contacting the involved mucosa of a patient who is immunocompromised or because of a planned course of chemotherapy or radiation therapy, is expected to become immunocompromised or is in a preclinical stage of mucositis where the symptoms have not become evident. The preventive method comprises contacting the affected mucosa with a composition which contains an amount of triclosan or a combination of triclosan and a cationic compound which is effective to prevent mucositis. The oral mucosa will usually be the area that is most affected by mucositis.

Generally the compositions used in the invention contain, about 0.01 to 5.3 wt % and preferably 0.1 to 0.5 wt % of triclosan and if a cationic compound is employed, about 0.01 to 0.3 wt % and preferably about 0.025 wt % of the cationic agent is added. Generally, semi-solid formulations will be formulated with higher levels of triclosan and the cationic agent. The amount of the formulation applied will depend upon the potential extent of the mucositis. Generally when a liquid formulation is applied for prevention of mucositis, from 5 ml to 30 ml is applied to the area of potential mucositis as a liquid with the patient being instructed to swallow, gargle or eject the excess amount of the formulation from the mouth, depending upon the area where the mucositis is to be prevented. If a semi-solid formulation is used, then a thin film can be applied to the area where the mucositis is to be prevented.

Generally it is preferred to initiate preventive therapy with the formulation of the invention prior to the appearance of clinical symptoms or prior to the initiation of an etiologic factor that may cause or is known to cause the appearance of mucositis. For example, the formulation would be administered prior to or concomitantly with the initiation of chemotherapy and/or radiation therapy, or up to 30 days prior to the start of radiation and/or chemotherapy. It is preferred to begin administration one to three days prior to the initiation of radiation and/or chemotherapy.

Administration of the formulation may be continued until the patient is no longer at risk of symptom manifestation, or any symptoms that may have occurred have been resolved. This includes administration of the formulation during the entire period when patients are at risk for mucositis, i.e. during radiation and/or chemotherapy and immediately thereafter. If symptoms occur, administration of the formulation should be continued for the purpose of suppressing or prevention of any exacerbation of symptoms. The formulation of Example 1 has been tested clinicaly by administering the formulation just prior to the initiation of chemotherapy and/or radiation therapy and has been able to reduce the occurence of mucositis symptoms from 90.2% in a group that received a vehicle control compared to 68.9% in the study group that received the formulation of Example 1 (p=0.015). A comparison of this test data with data from the literature has shown that the use of the formualtion of the invention was able to reduce the occurence of the most serious form of mucositis, i.e. ulcerative mucositis from 78.6% to 46.7%.

Triclosan is 2,4,4′-trichloro-2′-hydroxydiphenyl ether which is commercially available. The cationic agents include chlorhexidine and quaternary ammonium salts such as cetylpyridinium chloride (CPC) which is the monohydrate of the quaternary ammonium salt of pyridine and cetyl chloride. CPC is cationic, highly soluble in water and alcohol. Other cationic agents include benzalkonium chloride, benzethonium chloride, methylbenzethonium chloride and domiphen bromide. Chlorhexidine may be applied as the free base, or as the dihydrochloride or the gluconate salt.

The combination of triclosan and the cationic agent has the effect that the combined agents are readily adsorbed and retained on the mucosa while resisting removal by saliva and other fluids.

The compositions may be prepared as a liquid or a semi-solid formulation. The semi-solid compositions may vary from highly viscous liquids to gels or paste like formulations.

A liquid formulation may be prepared with purified water, the triclosan, the cationic agent and a solubilizer. The solubilizer may comprise a poloxamer. These materials are of the formula HO(CH₂CH₂O)ₐ(CH—(CH₃)(CH₂OH)_{b}(CH₂CH₂O)_{c}H where b is at least 15 and (CH₂CH₂O)ₐ+c is varied from 20 to 90% by weight and the weight average mol wt ranges from 10,000 to >16,000. The polyoxamers are available under the Pluronic trademark and Pluronic F127 is a preferred solubilizer. If solubilizer is employed, it will comprise from 0.5 to 8 wt % of the liquid composition. Generally, only liquid compositions in water will require a solubilizer; semi-solid formulations will not require the presence of a solubilizer.

A pharmaceutically acceptable alcohol such as ethyl alcohol may be optionally present as a cosolvent in an amount of 0.5 to 18% by weight.

However, the presence of as little as 3-10% by weight of ethyl alcohol can cause tissue irritation, a burning sensation or drying of the skin or the mucosa. The presence of ethyl alcohol in formulations is unacceptable for various patient groups including those with alcohol dependencies, liver dysfunction, and other metabolic disorders.

Preferred alcohol free formulations comprise the following ingredients (by weight):

| | | | |
|---|---|---|---|
| triclosan | 0.01-3.% | or | 0.1-1.0% |
| polyoxamers | 0.5-5% | or | 1-3% |
| polyhydric alcohol | 5-35% | or | 8-25% |
| water qs | 100% | | |

The compositions may also contain flavoring agents, coloring agents and the like.

The mucositis preventive formulation may include an anti-caries agent which is soluble in water such as sodium fluoride, stannous fluoride or sodium monofluorophosphate in an amount which is effective to inhibit tooth decay in an immunocompromised patient. Generally, this amount will be from 0.01 to 4% by weight, based on the weight of the fluoride ion. The amount may be varied depending on the particular source of the fluoride ion which is chosen. Certified color may be added in a minor amount e.g. 0.1% by weight. FD&C Blue No.1 or FD&C Yellow No.5 may be used as desired.

If desired, pharmaceutically acceptable zinc salts may be included in an amount of from 0.005 to 4% by weight in the formulation as a delivery enhancing agent, such as zinc citrate, zinc glycinate, zinc sulfate and the like.

The composition may also include triclosan and a copolymer of polyvinyl methyl ether with maleic anhydride or any other pharmaceutically acceptable delivery enhancing polymeric material. The amount of such polymer may vary from 0.05-4% by total weight of the composition.

### EXAMPLE 1

A typical liquid formulation will comprise:

| | % weight |
|---|---|
| triclosan | 0.100 |
| CPC | 0.024 |
| Sorbitol Solution, U.S.P. | 12.000 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.615 |

### EXAMPLE 2

A typical fluoridated liquid formulation will comprise:

| | % weight |
|---|---|
| triclosan | 0.100 |
| CPC | 0.024 |
| Sodium Fluoride | 0.020 |
| Sorbitol Solution, U.S.P. | 11.980 |
| Glycerin | 10.000 |
| Sodium Saccharin, U.S.P | 0.100 |
| Pluronic FI27, NF | 4.000 |
| 190 Proof Grain Alcohol, U.S.P. | 7.000 |
| Peppermint IFL2745 | 0.152 |
| Caramel Color AP100 | 0.0085 |
| Purified water | 66.615 |

### EXAMPLE 3

A typical semisolid formulation which is a cream:

will include:

| | | |
|---|---|---|
| triclosan | 0.1-5.3 | wt % |
| Cetaryl glucoside and cetaryl alcohol | 0.5-6.7 | wt % |
| (Emulgade PL 68/50, Henkel) | | |
| Cetaryl alcohol | 0.5-7.7 | wt % |
| (Lanette, Henkel) | | |
| Coco-Caprylate (Cedol LC, Henkel) | 0.5-6.0 | wt % |
| Dicapryl ether (Cetiet, Henkel) | 0.25-5.0 | wt % |
| Sweet almond oil | 0.25-5.0 | wt % |
| Petrolatum | 0.5-6.0 | wt % |
| Dimethicone (Silicone DC 200CS/Dow) | 0.1-5 | wt % |
| Phase B | | |
| CPC | 0.01-4.4 | wt % |
| glycerin | 0.5-4.6 | wt % |
| Sodium methylparaben/Sodium paraben | 0.01-0.03 | wt % |
| or | | |
| Sodium benzoate | 0.25-0.3 | wt % |
| Deionized water | 10-90 | wt % |

### EXAMPLE 4

A typical liquid formulation will comprise:

| | % weight |
|---|---|
| Triclosan | 0.200 |
| Sorbitol | 12.000 |
| Glycerin | 10.000 |
| Sodium Saccharin, U.S.P | 0.100 |
| Pluronic FI27, NF | 1.000 |
| Peppermint 1FL2745 | 0.152 |
| Caramel Color AP100 | 0.0085 |
| Purified water | qs 100.0 |

### EXAMPLE 5

A typical fluoridated liquid formulation will comprise:

| | % weight |
|---|---|
| Triclosan | 0.50 |
| Sodium Fluoride | 0.019 |
| Sorbitol | 12.000 |
| Glycerin | 10.000 |
| Sodium Saccharin, U.S.P | 0.100 |
| Pluronic FI27, NF | 1.000 |
| Peppermint 1FL2745 | 0.152 |
| Caramel Color AP100 | 0.0085 |
| Purified water | qs 100.00 |

### EXAMPLE 6

A typical liquid formulation containing a cationic agent will comprise:

| | % weight |
|---|---|
| Triclosan | 0.150 |
| Cetyl pyridinium chloride | 0.019 |
| Sorbitol | 12.000 |
| Glycerin | 10.000 |
| Sodium Saccharin, U.S.P | 0.100 |
| Pluronic FI27, NF | 1.000 |
| Peppermint 1FL2745 | 0.152 |
| Caramel Color AP100 | 0.0085 |
| Purified water | qs 100.00 |

### EXAMPLE 7

An example of a semi-solid formulation according to the invention is as follows:

| Phase A | | |
|---|---|---|
| triclosan | 0.3 | wt % |
| Cetaryl glucoside and cetaryl alcohol | 3.7 | wt % |
| (Emulgade FL 63/50, Henkel) | | |
| Cetaryl alcohol | 3.7 | wt % |
| (Lanette, Henkel) | | |
| Coco-Caprylate (Cedol LC, Henkel) | 3.0 | wt % |
| Dicapryl ether (Cetiet, Henkel) | 2.0 | wt % |
| Sweet almond oil | 2.0 | wt % |
| Petrolatum | 3.0 | wt % |
| Dimethicone (Silicone DC 200CS/Dow) | 0.6 | wt % |
| Phase B | | |
| CPC | 0.1 | wt % |
| Glycerin | 2.6 | wt % |
| Sodium methylparaben | 0.18 | wt % |
| Sodium paraben | 0.02 | wt % |
| Deionized water to | 100.0 | wt % |
| Phase C | | |
| Tocopheryl acetate (cophenol 1260/Henkel) | 1.0 | wt % |

The composition is prepared by separately heating Phase A and Phase B to 80° C. prior to forming these phases. Phase C is added with stirring at 55° C. until a smooth homogeneous mixture is obtained.

Weight percent is calculated as a percent of the total weight of all of the components.

The foregoing description of a preferred embodiment of the invention has been presented for purposes of illustration and description. It is not intended to be exhaustive or to limit the invention to the precise form disclosed. Obvious modifications or variations are possible in light of the above teachings. All such obvious modifications and variations are intended to be within the scope of the appended claims.

I claim:

1. A method of preventing mucositis, said method comprising applying to oral, pharyngeal, esophogeal, gastrointestinal and other mucosal tissues of the body an amount of composition which comprises triclosan or a combination of triclosan and a cationic agent in amounts which are effective to prevent the symptoms of mucositis.

2. A method of preventing mucositis as defined in claim 1 wherein the cationic agent is selected from the group consisting of chlorhexidine, cetylpyridium chloride, benzalkonium chloride, benzethonium chloride, methylbenzethonium chloride and domiphen bromide.

3. A method of preventing mucositis as defined in claim 1 wherein the cationic agent is cetylpyridium chloride.

4. A method of preventing mucositis as defined in claim 3 wherein the triclosan and cationic agent are combined in a liquid formulation.

5. A method of preventing mucositis as defined in claim 3 wherein the triclosan and the cationic agent are combined in a semi-solid formulation.

6. A method of preventing mucositis which comprises contacting the oral, pharyngeal, esophageal or gastrointestinal mucosa of a patient who is immunocompromised or because of a planned course of chemotherapy or radiation therapy, is expected to become immunocompromised and is predisposed to mucositis or is in a preclinical stage of mucositis where the symptoms have not become evident said method comprising contacting the affected area with an amount of composition which consists essentially of triclosan or a combination of triclosan and a cationic agent in amounts which are effective to prevent the symptoms of mucositis.

7. A method of preventing mucositis in a patient wherein the composition includes a fluoride.

8. A composition for preventing mucositis in an immunocompromised patient, said composition comprising a composition which comprises triclosan or a combination of triclosan and a cationic antibacterial agent in amounts which are effective to prevent the symptoms of mucositis and an amount of a fluoride compound which is effective to inhibit tooth decay in an immunocompromised patient.
